(19) United States
(12) Patent Application Publication
(10) Pub. No.: US 2023/0050588 A1
(43) Pub. Date: Feb. 16, 2023

(54) ANTIOXIDANT OR SKIN STRESS INHIBITOR

(71) Applicant: Shiseido Company, Ltd., Chuo-ku, Tokyo (JP)

(72) Inventors: Kazuyuki MIYAZAWA, Tokyo (JP); Renaud GILLET, Tokyo (JP); Bianca MCCARTHY, Tokyo (JP); Tetsuya KANEMARU, Tokyo (JP)

(73) Assignee: Shiseido Company, Ltd., Chuo-ku, Tokyo (JP)

(21) Appl. No.: 17/796,584

(22) PCT Filed: Jan. 29, 2021
(86) PCT No.: PCT/JP2021/003381

§ 371 Date: Jul. 29, 2022

(30) Foreign Application Priority Data
Jan. 31, 2020 (JP) 2020-015642

(51) Int. Cl. A61N 5/06; A61K 8/64; A61K 8/67; A61K 8/27; A61Q 17/04

(52) U.S. Cl. CPC A61N 5/062; A61K 8/645; A61K 8/673; A61K 8/27; A61Q 17/04; A61N 2005/0661

(57) ABSTRACT

An anti-oxidizing agent or skin stress suppressing agent was provided with.

An anti-oxidizing agent or skin stress suppressing agent comprising a wavelength conversion substance as an active ingredient; a composition and a product comprising the anti-oxidizing agent or skin stress suppressing agent; and a method for suppressing oxidization and skin stress of skin using thereof were provided with. The present invention can exhibit a desirable effect on skin by effectively making use of ultraviolet light to suppress skin cells.

### FIELD

The present invention relates to an anti-oxidizing agent or skin stress suppressing agent comprising a wavelength conversion substance, to a composition and product comprising the anti-oxidizing agent or skin stress suppressing agent, and to a method for suppressing oxidization and skin stress in skin using the same.

### BACKGROUND

The harm to skin caused by ultraviolet light includes adverse effects such as skin cancer, photoaging, skin spots, wrinkles and inflammation, which are also undesirable from the viewpoint of health and beauty.

Many measures are therefore being taken to protect the skin from ultraviolet light. Such measures include the use of sunscreens, the implementation of indoor spaces for avoidance of sunlight, and the use of head coverings and clothing treated to block UV rays and films designed to block UV rays.

### CITATION LIST

### Patent Literature

[PTL 1] Japanese Patent Publication No. 6424656

[PTL 2] Japanese Patent Publication No. 6361416

[PTL 3] International Patent Publication No. 2018/004006

[PTL 4] Japanese Unexamined Patent Publication No. 2018-131422

[PTL 5] Japanese Unexamined Patent Publication HEI No. 5-117127

[PTL 6] Japanese Patent Publication No. 4048420

[PTL 7] Japanese Patent Publication No. 4677250

[PTL 8] Japanese Patent Publication No. 3303942

[PTL 9] Japanese Unexamined Patent Publication No. 2017-88719

[PTL 10] International Patent Publication No. 2018/117117

### SUMMARY

### Technical Problem

It is an object of the present invention to provide a novel anti-oxidizing agent or skin stress suppressing agent that utilizes conversion of wavelength of ultraviolet light.

### Solution to Problem

The present inventors have conducted active research with the aim of allowing ultraviolet light to be effectively utilized on skin. As a result, an anti-oxidizing agent or skin stress suppressing agent has been devised by finding that the expression of oxidative stress related substances is changed by irradiating ultraviolet light to skin cells through wavelength conversion substance that converts ultraviolet light wave length.

The present application provides the present invention with the aspects set forth below.

(1) A anti-oxidizing agent or skin stress suppressing agent comprising a wavelength conversion substance as an active ingredient, wherein the wavelength conversion substance converts the wavelength of ultraviolet light contained in incident light to emit emission light having a wavelength longer than the wavelength of the ultraviolet light.

(2) The anti-oxidizing agent or skin stress suppressing agent according to (1), wherein the ultraviolet light has a peak wavelength between 200 nm and 400 nm.

(3) The anti-oxidizing agent or skin stress suppressing agent according to (1) or (2), wherein the emission light has a peak wavelength between 450 nm and 700 nm.

(4) The anti-oxidizing agent or skin stress suppressing agent according to any one of (1) to (3), wherein the wavelength conversion substance comprises one or more phycobiliproteins selected from among allophycocyanin, C-phycocyanin, R-phycocyanin, phycoerythrocyanin, B-phycoerythrin, b-phycoerythrin, C-phycoerythrin and R-phycoerythrin; one or more inorganic phosphors selected from among zinc oxide phosphors, magnesium titanate phosphors and calcium phosphate phosphors; one or more components selected from among vitamin A, β-carotene, vitamin K, vitamin B1, vitamin B2, vitamin B6, vitamin B12, folic acid, niacin, lycopene, gardenia, safflower, turmeric, cochineal, perilla, red cabbage, flavonoids, carotenoids, quinoids, porphyrins, anthocyanins, and polyphenols; and/or one or more pigments selected from among Red No. 401, Red No. 227, Red No. 504, Red No. 218, Orange No. 205P, Yellow No. 4, Yellow No. 5, Green No. 201, Pyranin Conch, Blue No. 1, 2,4-diaminophenoxyethanol hydrochloride, Arizulin Purple SS, Violet No. 401, Black No. 401, Helindone Pink, Yellow No. 401, Bentizine Yellow G, Blue No. 404, Red No. 104, and meta-aminophenol.

(5) The anti-oxidizing agent or skin stress suppressing agent according to (4), wherein the wavelength conversion substance comprises one or more phycobiliproteins selected from among allophycocyanin, C-phycocyanin, R-phycocyanin, phycoerythrocyanin, B-phycoerythrin, b-phycoerythrin, C-phycoerythrin and R-phycoerythrin; one or more inorganic phosphors selected from among zinc oxide phosphors, magnesium titanate phosphors and calcium phosphate phosphors; and/or one or more B vitamins selected from among vitamin B1, vitamin B2, vitamin B6 and vitamin B12.

(6) A composition comprising the anti-oxidizing agent or skin stress suppressing agent according to any one of (1) to (5).

(7) The composition according to (6), wherein the composition is a skin external composition for suppressing oxidization and skin stress in skin by exposing skin to light containing ultraviolet light.

(8) A cosmetic method for suppressing oxidization and skin stress of a subject, comprising: applying the composition according to any one of (6) to (9) to skin of a subject, and exposing the composition-applied skin with light containing ultraviolet light.

(9) A product comprising the anti-oxidizing agent or skin stress suppressing agent according to any one of (1) to (5).

(10) The product according to (9), wherein the product is for suppressing oxidization and skin stress in skin by exposing the skin to light passing through the product, which contains ultraviolet light.

(11) A cosmetic method for suppressing oxidization and skin stress in skin of a subject, comprising:

bringing light containing ultraviolet light through the product according to (9) or (10), and exposing the skin of the subject to light passing through the product.

### Advantageous Effects of Invention

The present invention can suppress oxidization and/or skin stress in skin cells by effectively making use of ultraviolet light, and is based on the finding that suppression of oxidization and/or skin stress can results in a desirable effect on skin. The invention provides novel uses of the aforementioned compounds that have conventionally been used primarily as dyes, pigments, ultraviolet scattering agents, ultraviolet absorbers, nutrients and antioxidants. The invention also helps to improve quality of life by providing a more positive feeling for persons who have attempted to avoid ultraviolet light as much as possible for beauty or health reasons when outdoors.

### BRIEF DESCRIPTION OF DRAWINGS

FIG. 1 is a schematic diagram illustrating Experiment 1.

### DESCRIPTION OF EMBODIMENTS

The anti-oxidizing agent or skin stress suppressing agent of the invention comprises a wavelength conversion substance as an active ingredient. A wavelength conversion substance is a substance that converts the wavelength of ultraviolet light contained in incident light and emits emission light having a wavelength longer than the wavelength of the ultraviolet light.

The ultraviolet light may include UVA, UVB and UVC. According to one embodiment, the ultraviolet light is light with a peak wavelength of 200 nm to 400 nm. The ultraviolet light may also be included in incident light such as sunlight, for example. Alternatively, the incident light may be ultraviolet light, and artificially generated ultraviolet light may be used. Ultraviolet light can have various effects on the skin. As an example, ultraviolet light is known to cause sunburns, such as sunburn and suntan and to produce reactive oxygen species and to cause DNA damage.

The emission light emitted by the wavelength conversion substance has a longer wavelength than ultraviolet light, with a peak wavelength of preferably 500 nm to 700 nm. The emission light may have one or more peaks at 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, 600 nm, 610 nm, 620 nm, 630 nm, 640 nm, 650 nm, 660 nm, 670 nm, 680 nm, 690 nm or 700 nm, or in any range within these values, though without being restrictive, or it may be red light, orange light, green light or blue light. According to one embodiment, the wavelength conversion substance has its main wavelength at 450 nm to 700 nm, for example 500 nm to 700 nm, for light emitted upon excitation with excitation light of 200 nm to 400 nm.

Examples of wavelength conversion substances include the following components:

phycobiliproteins such as allophycocyanin, C-phycocyanin, R-phycocyanin, phycoerythrocyanin, B-phycoerythrin, b-phycoerythrin, C-phycoerythrin and R-phycoerythrin;

natural or synthetic components such as vitamin A, n-carotene, vitamin K, vitamin B1, vitamin B2, vitamin B6, vitamin B12, folic acid, niacin, lycopene, gardenia, safflower, turmeric, cochineal, perilla, red cabbage, flavonoids, carotenoids, quinoids, porphyrins, anthocyanins, polyphenols, dyes such as Red No. 401, Red No. 227, Red No. 504, Red No. 218, Orange No. 205 P, Yellow No. 4, Yellow No. 5, Green No. 201, Pyranin Conch, Blue No. 1, 2,4-diaminophenoxyethanol hydrochloride, Arizulin Purple SS, Violet No. 401, Black No. 401, Helindone Pink, Yellow No. 401, Bentizine Yellow G, Blue No. 404, Red No. 104, meta-aminophenol; and phosphors obtained by fluorescent-doping of inorganic compounds, for example, blue phosphors comprising the amorphous silica particles mentioned in Japanese Patent

No. 6424656, cerium and phosphorus and/or magnesium, and red phosphors comprising compounds obtained by europium activation of mixed crystals consisting of the alkaline earth metal sulfides described in Japanese Patent No. 6361416 combined with gallium compounds, the zinc oxide phosphors mentioned in International Patent Publication No. 2018/004006, the zinc oxide phosphors mentioned in Japanese Unexamined Patent Publication No. 2018-131422, and the inorganic phosphors mentioned in Japanese Unexamined Patent Publication HEI No. 5-117127. According to one embodiment, the inorganic phosphor is one or more phosphors selected from among phosphors obtained by doping zinc oxides represented by ZnO: Zn, Zn_{1+z}, ZnO₁₋ₓ with the sulfur-containing compounds mentioned in International Patent Publication No. 2018/004006, including sulfides and/or sulfates such as zinc sulfide or zinc sulfate, magnesium titanate phosphors obtained by doping magnesium titanates such as MgTiO₃ or Mg₂TiO₄ with manganese, and calcium phosphate phosphors obtained by doping calcium phosphates such as Ca(H₂PO₄)₂, CaHPO₄ or Ca₃(PO₄)₂ with cerium.

The wavelength conversion substance may be obtained by extraction from a natural source such as an animal, plant or algae, or it may be obtained by an artificial method such as chemical synthesis. For example, phycobiliproteins can be prepared by extraction from algae, including blue-green algae such as spirulina (Spirulina platensis) or red algae such as porphyridiophylla (Porphyridium purpureum), by the method described in Japanese Patent No. 4048420, Japanese Patent No. 4677250 or Japanese Patent No. 3303942, for example. Zinc oxide phosphors can be produced by the method described in International Patent Publication No. 2018/004006, Japanese Unexamined Patent Publication No. 2018-131422 or Japanese Unexamined Patent Publication HEI No. 5-117127, for example. Magnesium titanate phosphors can be produced by the method described in Japanese Unexamined Patent Publication No. 2017-88719. Calcium phosphate phosphors can be produced by the method described in International Patent Publication No. 2018/117117.

So long as the wavelength conversion effect of the invention is not impaired, these wavelength conversion substances may be composed of, or may include, the components mentioned above, and they may be single components alone or combinations of more than one of the components. For example, the aforementioned phycobiliproteins or inorganic material phosphors may be mixed with other wavelength conversion substances such as B vitamins (vitamin B1, vitamin B2, vitamin B6 or vitamin B12) to exhibit synergistic effects. These components are merely examples, however, and any other substances that exhibit the wavelength conversion effect of the invention may be used.

The wavelength conversion substance content in the anti-oxidizing agent or skin stress suppressing agent, composition or product of the invention is not particularly restricted so long as the wavelength conversion effect of the invention is not impaired, and it may be appropriately determined for the type of wavelength conversion substance and the purpose of use of the anti-oxidizing agent or skin stress suppressing agent or composition. It may be any content in the range of 0.01 to 99.99 wt % or 0.1% to 999 wt %, for example.

Skin stress refers to oxidative stress received by cells of the skin, especially of the epidermis and dermis. Cells in the skin are generally damaged by reactive oxygen species (ROS), leading to inflammation and reduced skin barrier function. Therefore, a condition of high skin stress refers to a condition in which skin cells accumulate damage due to reactive oxygen species. Reactive oxygen species are derived from abnormal reactions of the cellular electron transport chain and from ultraviolet radiation. When the anti-oxidizing agent or skin stress suppressing agent of the present invention is irradiated with ultraviolet rays, an emission light is emitted, and the emission light changes the expression of the oxidative stress-related proteins in the skin cells, whereby the antioxidant action and/or the skin stress suppressing action is exhibited. The oxidizing stress related proteins include AhR, AhRR, CYP1B1, and HMOX1, etc. The anti-oxidizing effect and/or skin stress suppressing effect may be an inhibitory effect of oxidative and/or skin stress induced by ultraviolet light, or an inhibitory effect of oxidative and/or skin stress induced by causes other than ultraviolet light. The anti-oxidizing agent or skin stress suppressing agent of the present invention may be used in any subject, and may be applied to a subject subjected to ultraviolet light outside, or to a subject subjected to oxidative stress and/or skin stress.

Aromatic hydrocarbon receptors (AhRs) are transcription factors belonging to bHLH-PAS(Basic Helix-Loop-Helix-Per-Arnt-Sim) family. It is also known that AhR is activated by aromatic hydrocarbons such as dioxins, which become a ligand, and translocates into the nucleus, and functions as a transcription factor, while AhR is activated by UV. When AhR is activated, the expression of enzymes involved in xenobiotic metabolism, including the drug-metabolizing enzymes CYP1 and the glutathione S-transferase-Y subunit are increased, and pathways involved in ROS accumulation are activated. ROS accumulation leads to oxidative stress, which results in production of inflammatory cytokine as well as oxidative DNA damage.

The aromatic hydrocarbon receptor repressor (AHRR) functions as a repressor which suppresses the action of AhR. When the expression of AHRR is increased, the activity of AhR is reduced, whereby drug metabolism pathways and ROS-accumulation pathways are suppressed.

Cytochrome P4501B1(CYP1B1) is an enzyme belonging to the cytochrome P450 superfamily and is an enzyme involved in drug metabolism of polycyclic aromatic hydrocarbons, etc. UV irradiation results in increased expression of CYP1B1in keratinocytes. CYP1B1 is also activated by AHR and induces oxidative stress pathways.

Heme oxygenase 1 (HMOX1) is an enzyme involved in the initial step of heme metabolism. HMOX1 degrades heme into biliverdin. Heme is a prosthetic group essential for biological activity, but heme released from proteins becomes a deleterious molecular that generates reactive oxygen. Therefore, the activation of HMOX1 contributes to the reduction of oxidative stress and anti-inflammation. HOMX1 is induced by oxidative stress such as UV-irradiation.

Any form of administration may be used for the anti-oxidizing agent or skin stress suppressing agent and composition of the invention, but an external preparation for skin will often be preferred, such as a drug, quasi drug or cosmetic, for suppressing oxidization and skin stress in skin by exposing the skin to light containing ultraviolet light. When the anti-oxidizing agent or skin stress suppressing agent or composition of the invention is to be used as an external preparation for skin, the dosage form, coating method and number of doses may be determined as desired. For example, it may be applied onto skin in the form of cosmetic water or a spray, oil, cream, latex, gel, sunscreen or suntan lotion either periodically or irregularly, once or several times per day at morning, noon or evening, or before going out or engaging in outdoor activities, marine sports or skiing, for example, when exposure to sunlight is expected.

The anti-oxidizing agent or skin stress suppressing agent and composition of the invention may also be used in combination with an additive such as an excipient, preservative, thickener, binder, disintegrator, dispersing agent, stabilizer, gelling agent, antioxidant, surfactant, preservative, oil, powder, water, alcohol, thickener, chelating agent, silicone, antioxidant, humectant, aromatic, drug component, antiseptic agent, pH adjustor or neutralizer, selected as necessary or desired. It may also be used in combination with other anti-oxidizing agent or skin stress suppressing agents to increase the effect of the invention.

The present invention further provides products such as sun visors, caps, clothing, gloves, screen films, window sprays or creams, window materials or wall materials, for example, that comprise a anti-oxidizing agent or skin stress suppressing agent of the invention and are intended to inhibit oxidization and/or skin stress in skin. The usage of additives in the products of the invention and the forms of the products may also be as desired.

The present invention further provides a method for producing the anti-oxidizing agent or skin stress suppressing agent, composition or product of the invention. A method for suppressing oxidization and skin stress in skin of a subject is also provided, the method comprising application of a anti-oxidizing agent or skin stress suppressing agent or composition of the invention onto the skin of a subject and exposing the skin to light containing ultraviolet light after application of the anti-oxidizing agent or skin stress suppressing agent or composition; or passing light containing ultraviolet light through the product of the invention, and exposing the skin to the transmitted light; wherein the anti-oxidizing agent or skin stress suppressing agent, composition or product converts the wavelength of ultraviolet light in the incident light and emits emission light with a longer wavelength than the wavelength of the ultraviolet light, transmitting the ultraviolet light with a peak wavelength of preferably 200 nm to 400 nm as light with a peak wavelength of 450 nm to 700 nm, for example 500 nm to 700 nm. The method for suppressing oxidization and skin stress in skin of a subject will often be for the purpose of beautifying, instead of treatment by a doctor or health care professional. The invention further provides a cosmetic counseling method for supporting cosmetology, which includes providing a cosmetic method, anti-oxidizing agent or skin stress suppressing agent, composition or product of the invention to a subject.

### EXAMPLES

The present invention will now be explained in greater detail by examples. However, the invention is in no way limited by the examples.

### Experiment 1: Change in Gene Expression by Applying Wavelength Conversion Substances

### Experiment 1-1: Preparation of Wavelength Conversion Substances

Wavelength conversion substances were prepared in the following manner.

### (1) C-phycocyanin

C-phycocyanin is obtained from spirulina (Spirulina platensis) extract, the absorption spectrum having a peak wavelength at 350 nm and the emission spectrum having peak wavelengths at 640 nm and 700 nm.

### (2) Ribovlavin (Vitamin B2)

Riboflavin is also referred to as vitamin B2, the absorption spectrum having a peak wavelength at 445 nm and the emission spectrum having peak wavelengths at 530 nm.

### (3) Zinc Oxide Phosphor

Lumate G by Sakai Chemical Industry Co., Ltd. was used. Lumate G is a zinc oxide phosphor obtained by doping ZnO with a sulfur-containing compound and then firing as described in International Patent Publication No. 2018/004006, the absorption spectrum having a peak wavelength at 365 nm and the emission spectrum having a peak wavelength at 510 nm.

### (4) Magnesium Titanate Phosphor

Lumate R by Sakai Chemical Industry Co., Ltd. was used. Lumate R is a magnesium titanate phosphor obtained by doping MgTiO₃ with manganese, the absorption spectrum having a peak wavelength at 365 nm and the emission spectrum having peak wavelengths in the range of 660 to 680 nm.

The wavelength conversion substances of (1) and (2) were dissolved in water to prepare solutions at concentrations of 1% and 5%.

The wavelength conversion substances of (3) and (4) were dispersed in alcohol to prepare 5% and 10% dispersions.

### Experiment 1-2: Preparation of Cell Samples

Cell samples were prepared in the following manner.

1. Normal Human Epidermal Keratinocytes (PromoCell) were used. A cell suspension (1 mL) stored with liquid nitrogen was placed in a hot water bath (37° C.) and thawed until small ice pellets remained, and then diluted with 9 mL of warm KGM medium.
2. The diluted suspension was gently mixed and transferred to a T75 flask, and incubated overnight at 37° C.
3. On the following day, the medium was exchanged with 10 mL of fresh medium.
4. The medium was periodically exchanged (once every 2 to 3 days), while continuing growth of the cells. During this time, a microscope was used to observe the cells and to confirm that the cells had proliferated with the proper form.
5. Once the cells reached approximately 80% confluence, they were subcultured.
6. Subculturing of the cells was carried out by rinsing and aspirating once in 10 mL of warm PBS.
7. 5 mL of warm trypsin was added to the T75 flask to cover the bottom of the flask with the trypsin solution, and the mixture was aspirated after standing for 1 minute at room temperature.
8. The flask was set in an oven at 37° C. for 5 minutes (maximum) for keratinocytes. A microscope was used to observe the cells, confirming that they were small and elliptical.
9. The sides of the T75 flask were then lightly tapped to free the cells. A microscope was used to observe the cells and confirm that they were freely moving.
10. The keratinocytes were resuspended in 5 mL of warm trypsin neutralizing solution, and transferred to a sterilized 50 mL Falcon tube. The flask was further rinsed with 5 mL of warm FGM and added to the Falcon tube, to ensure transfer of all of the cells.
11. The cells were centrifuged at 10,000 rpm for 5 minutes (4° C.), and the supernatant was carefully removed while avoiding disturbing the cell pellet.
12. The keratinocytes were resuspended in KGM at a concentration of 4×10⁴ cells/well (500 μL), and plated in collagen coated glass bottom 4-well chamber slide.
13. The medium was replaced every two or three days, and cells were proliferated until they reached 60 to 70% confluence (differing for the type of experiment).
14. At 24 hours before irradiation, the medium was changed to non-supplemented medium.

### Experiment 1-3: Ultraviolet Light Irradiation

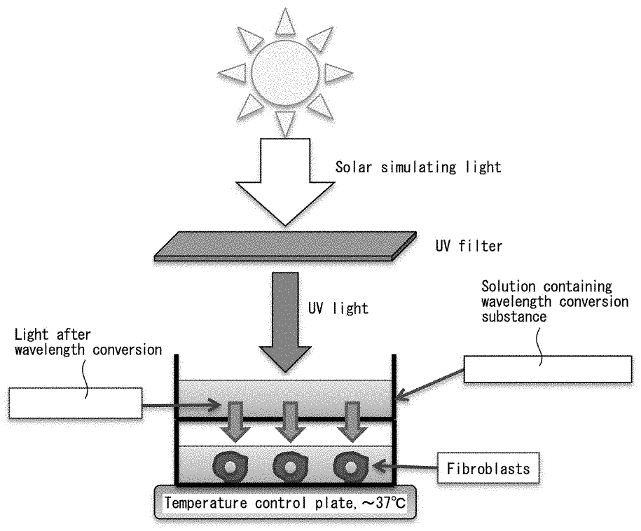

1. At least 30 minutes prior to irradiation, the power source of a solar simulator was activated to warm up the lamp. The solar simulator used was a UG11 filter. A UG11 filter is a filter that allows passage of UVB alone while cutting light of other wavelengths. The UV light passing through the UG11 filter had a peak wavelength of 300 nm to 385 nm.
2. The temperature control plate was turned on and set to 33° C.
3. The cells prepared in Experiment 1-2 were rinsed once with warm PBS.
4. A 0.5 mL portion of warmed Martinez solution (145 mM NaCl, 5.5 mM KCl, 1.2 mM MgCl₂.6H₂O, 1.2 mM NaH₂PO₄.2H₂O, 7.5 mM HEPES, 1 mM CaCl₂ and 10 mM D-glucose) was added to each well.
5. As shown in FIG. 1, the cell-containing wells were set on a plate, 0.4 ml of each solution containing the wavelength conversion substances (1)-(4) prepared in Experiment 1-1 was injected into the wells of a 24-well plate, the cell-containing wells were placed over it in a manner covering the wells, and UV light was irradiated into the cell solution through the wavelength conversion substance solution without allowing direct contact between the wavelength conversion substance solution and the cell solution.
6. The irradiation was carried out to a total radiation dose of 100 mJ/cm². As controls, there were prepared a sample of the cells directly irradiated with UV light without setting a wavelength conversion substance plate on the cell-containing wells, and a sample of the cells cultured in a dark environment without irradiation of UV light.
7. After irradiation, the Martinez solution was exchanged with warmed KGM (supplement-free), and the plate was returned to the 37° C. incubator, and incubated for 24 hours.

### Experiment 2: Microarray

### Experiment 2-1: RNA Extraction

Cell samples incubated for 24 h after irradiating ultraviolet light in Experiments 1-3 were washed with 500 μl warm PBS, and PBS was completely aspirated. Qiagen RNeasy Mini Kit prep (Qiagen, 74106) were used to extract RNA according to the product instructions.

### Experiment 2-2: Microarray

Microarray for Human gene-expression (SurePrint G3 Human GE Microarray 8×60K Ver. 3.0 (Agilent technology)) was used to perform the analysis of RNAs extracted in Experiment 2-1. The extracted RNA was subjected to labeling reactions, amplification reactions, purification, and quantitation of cRNA according to the protocol provided by Agilent Technology to prepare hybridization samples. Microarrays were observed with AGILINT C MICROARRAY SCANNER to identify genes whose expression was significantly reduced or increased by the presence or absence of wavelength-converting material, and be shown below.

TABLE 1

| Gene Name | Lina Blue | Vitamin B2 | Lumate G | Lumate R |
|---|---|---|---|---|
| AhR | ○ | ○ | x | ○ |
| CYP1B1 | ○ | ○ | x | ○ |
| AHRR | ○ | ○ | x | ○ |
| HMOX1 | ○ | x | ○ | x |

○ corresponds to genes whose expression is significantly changed,
x corresponds to genes whose expression is not changed. The expression of AhR and CYP1B1 is reduced, and the express

These results indicate that UV-irradiation against wavelength-conversion substances results in change in gene expression of oxidizing and/or skin stress related proteins, whereby the effect of suppressing oxidative and/or skin stress is exerted.

The embodiments of the invention described above are not intended to place limitations on the invention, and various modifications including cosmetics and drug compositions may be incorporated, which fall within the gist of the invention.

1. A anti-oxidizing agent or skin stress suppressing agent comprising a wavelength conversion substance as an active ingredient,
wherein the wavelength conversion substance converts the wavelength of ultraviolet light contained in incident light to emit emission light having a wavelength longer than the wavelength of the ultraviolet light.

2. The anti-oxidizing agent or skin stress suppressing agent according to claim 1, wherein the ultraviolet light has a peak wavelength between 200 nm and 400 nm.

3. The anti-oxidizing agent or skin stress suppressing agent according to claim 1 or 2, wherein the emission light has a peak wavelength between 450 nm and 700 nm.

4. The anti-oxidizing agent or skin stress suppressing agent according to any one of claims 1 to 3, wherein the wavelength conversion substance comprises one or more phycobiliproteins selected from among allophycocyanin, C-phycocyanin, R-phycocyanin, phycoerythrocyanin, B-phycoerythrin, b-phycoerythrin, C-phycoerythrin and R-phycoerythrin; one or more inorganic phosphors selected from among zinc oxide phosphors, magnesium titanate phosphors and calcium phosphate phosphors; one or more components selected from among vitamin A, β-carotene, vitamin K, vitamin B1, vitamin B2, vitamin B6, vitamin B12, folic acid, niacin, lycopene, gardenia, safflower, turmeric, cochineal, perilla, red cabbage, flavonoids, carotenoids, quinoids, porphyrins, anthocyanins, polyphenols, and/or one or more pigments selected from among Red No. 401, Red No. 227, Red No. 504, Red No. 218, Orange No. 205P, Yellow No. 4, Yellow No. 5, Green No. 201, Pyranin Conch, Blue No. 1, 2,4-diaminophenoxyethanol hydrochloride, Arizulin Purple SS, Violet No. 401, Black No. 401, Helindone Pink, Yellow No. 401, Bentizine Yellow G, Blue No. 404, Red No. 104, meta-aminophenol.

5. The anti-oxidizing agent or skin stress suppressing agent according to claim 4, wherein the wavelength conversion substance comprises one or more phycobiliproteins selected from among allophycocyanin, C-phycocyanin, R-phycocyanin, phycoerythrocyanin, B-phycoerythrin, b-phycoerythrin, C-phycoerythrin and R-phycoerythrin; one or more inorganic phosphors selected from among zinc oxide phosphors, magnesium titanate phosphors and calcium phosphate phosphors; and/or one or more B vitamins selected from among vitamin B1, vitamin B2, vitamin B6 and vitamin B12.

6. A composition comprising the anti-oxidizing agent or skin stress suppressing agent according to any one of claims 1 to 5.

7. The composition according to claim 6, wherein the composition is a skin external composition for suppressing oxidization and skin stress in skin by exposing skin to light containing ultraviolet light.

8. A cosmetic method for suppressing oxidization and skin stress of a subject, comprising:
applying the composition according to any one of claims 6 to 9 to skin of a subject, and
exposing the composition-applied skin with light containing ultraviolet light.

9. A product comprising the anti-oxidizing agent or skin stress suppressing agent according to any one of claims 1 to 5.

10. The product according to claim 11, wherein the product is for suppressing oxidization and skin stress in skin by exposing the skin to light passing through the product, which contains ultraviolet light.

11. A cosmetic method for suppressing oxidization and skin stress in skin of a subject, comprising:
passing through light containing ultraviolet light through the product according to claim 9 or 10, and
exposing the skin of the subject to light passing through the product.
